(19) United States
(12) Patent Application Publication
(10) Pub. No.: US 2026/0207190 A1
(43) Pub. Date: Jul. 23, 2026

(54) SUTURE DEVICE WITH MOVABLE MECHANISM AND LOCKING FEATURE

(71) Applicant: Adam RANELLONE, Putnam Valley, NY (US)

(72) Inventors: Adam Ranellone, Putnam Valley, NY (US); David Michael Uhlenhake, Toledo, OH (US); Damion Vitt, Fostoria, OH (US)

(21) Appl. No.: 19/143,267

(22) PCT Filed: Dec. 22, 2023
(86) PCT No.: PCT/US2023/085671

§ 371 Date: Jun. 25, 2025

Related U.S. Application Data
(60) Provisional application No. 63/435,458, filed on Dec. 27, 2022.

(51) Int. Cl. A61B 17/04; A61B 17/00

(52) U.S. Cl. CPC A61B 17/0467; A61B 17/0483; A61B 2017/00367; A61B 2017/00424; A61B 2017/00477

(57) ABSTRACT

The present invention relates to a suture device (100) including a first jaw (112) and a second jaw (114) connected by a. pivot (116), and an arm (102) connected to the second jaw (114) and the pivot (102) . The device (100) further includes a moving mechanism (108) that can move between an open position (151) and a closed position (152). The moving mechanism (108) includes a button (124) that compresses the arm (102), a support block (126) that supports the button (124) and moves within a support ledge (128) to allow the moving mechanism (108) to open (151) and dose (152), and a locking ledge (130) that maintains the position of a. hook (132) on the button (124) to lock the suture device (100) in the closed position (152). This suture device (100) provides improved control and ease of use during surgical procedures.

### CROSS-REFERENCE TO RELATED APPLICATIONS

This application claims the benefit of U.S. Provisional Application No. 63/435,458, filed on Dec. 27, 2022. The entire disclosure of the above application is incorporated herein by reference.

### FIELD

The present technology relates to the field of medical devices, specifically disposable, handheld surgical tools for suturing wounds.

### INTRODUCTION

This section provides background information related to the present disclosure which is not necessarily prior art.

Various suture devices have been developed to facilitate the process of suturing wounds or incisions. These devices typically include a pair of jaws that hold the suture thread and a mechanism to close the jaws and secure the suture in place. However, existing suture devices often suffer from limitations in terms of ease of use, precision, and reliability.

One approach in the prior art involves using a simple hinge mechanism to connect the jaws of the suture device. While this design allows for basic opening and closing of the jaws, it lacks the ability to provide precise control over the closing force and can not securely hold the suture in place. Additionally, the lack of a locking mechanism in these devices can result in accidental opening of the jaws during the suturing process, leading to potential complications.

Another approach in the prior art involves incorporating a spring mechanism to provide the closing force for the jaws. While this design allows for more controlled and consistent closing force, it can still lack the ability to securely lock the suture device in the closed position. The absence of a reliable locking mechanism can result in unintended release of the suture, compromising the integrity of the wound closure.

Furthermore, some existing suture devices utilize complex and bulky mechanisms to achieve the desired functionality. These devices can be difficult to handle and maneuver, particularly in tight or confined spaces. The complexity of these mechanisms can also increase the risk of mechanical failure or malfunction during use.

Traditional suturing of body tissues is a time-consuming aspect of most surgical procedures and general medical care. The standard set of instruments commonly found in a suture kit used to assist certified medical professionals, such as physicians, nurse practitioners, and physician assists, with suturing a patient can include a needle driver, toothed and nontoothed forceps, suturing scissors, and suturing material.

Although these instruments are standard within the field of medicine, continued use of these instruments can cause numerous problems. For example, the intended gripping method of the needle driver can cause the certified medical professional to experience soreness in the fingers. Suturing a patient can be a time consuming and repetitive process and, as such, holding the needle driver for an extended period of time can cause hand cramping and pain.

Further, as needle drivers lack hand grips, there is no added padding or comfort provided for the user. Finger loops present in a typical needle driver can transfer forces to the user's fingers and knuckles, in use, which can create narrow pressure points resulting in soreness and fatigue.

As another example, current suturing devices can be highly inefficient. The design of the typical needle driver does not allow for full wrist torsion and, therefore, limits the user's range of motion during suturing. Further, the design of the needle drivers can require that the user drop or remove their hand from the device to use a separate tool, such as suturing scissors, to cut the suture material. The hinge of the needle driver can also frequently snag the suture material, in use.

Therefore, none of these approaches have provided a comprehensive solution that combines the features described in this disclosure.

Accordingly, there is a continuing need for a suture device and method that solves multiple problems with standard disposable needle drivers and suture scissors used for wound closure. Standard devices cause hand and finger soreness during prolonged use due to non-ergonomic grips and wrist motion. Drying blood also increases difficulty of use over time.

Additionally, standard tools require repeatedly putting down and picking up separate needle drivers and scissors, costing valuable time during procedures. The present invention incorporates both cutting and needle driving features into a single ergonomic device to address these problems. Specifically, the enhanced grip, locking mechanism, and built-in scissors improve comfort, control, and efficiency compared to existing disposable suturing tools.

### SUMMARY

In concordance with the instant disclosure, the present invention solves multiple problems with standard disposable needle drivers and suture scissors used for wound closure. Standard devices cause hand and finger soreness during prolonged use due to non-ergonomic grips and wrist motion. Drying blood also increases difficulty of use over time. Additionally, standard tools require repeatedly putting down and picking up separate needle drivers and scissors, costing valuable time during procedures. The present invention incorporates both cutting and needle driving features into a single ergonomic device to address these problems.

Specifically, the enhanced grip, locking mechanism, and built-in scissors improve comfort, control, and efficiency compared to existing disposable suturing tools, has surprisingly been discovered.

The present technology includes articles of manufacture, systems, and processes that relate to a suture device for reliable and ergonomic suturing. The suture device includes a first jaw and a second jaw connected by a pivot and disposed adjacent to one another to form a pair of jaws. The pair of jaws contains a cutting portion formed by a first cutting surface on the first jaw and a second cutting surface on the second jaw that can cooperate to cut material such as suture. A moving mechanism is disposed adjacent to an arm connected to the second jaw and pivot. The moving mechanism includes a button, support block, support ledge, and locking ledge. The button compresses the arm when depressed and can slide forwards and backwards. Sliding the button back causes a hook on the button to contact the locking ledge which locks the suture device in a closed position.

Further, the moving mechanism and button allow the pair of jaws to move between an open position and closed position. In the open position, a spring adjacent to the arm is relaxed allowing the jaws to open. By depressing the button, the spring tensions thereby moving the second jaw and closing the jaws. Advantageously, when closed the pair of jaws can grasp objects such as needles.

Additionally, the suture device contains an ergonomic hand grip that allows a user to comfortably hold the device for extended periods without hand cramping. This provides improved ergonomics compared to standard needle drivers. In another embodiment, a sliding mechanism between the second jaw and support ledge locks the device open or closed.

The suture device can be formed from plastic materials allowing for disposability after use. Further, the button can be positioned along the length of the device to allow for optimal ergonomics when moving between open and closed positions. This allows for improved control and usability compared to standard devices.

Overall, the suture device provides reliable suturing through its cutting portion and jaw design. The ergonomic shape and moving mechanism also improve comfort and locking ability compared to standard needle drivers. These advantages allow for precise suturing and reduced hand fatigue.

In one embodiment, the techniques described herein relate to a suture device. The suture device includes: a first jaw and a second jaw disposed adjacent to one another and connected by a pivot; an arm connected to the second jaw and the pivot; a moving mechanism disposed adjacent to the arm and configured to move between an open position and a closed position. The moving mechanism includes: a button disposed adjacent to and in contact with the arm, the button configured to compress the arm; a support block disposed adjacent to a support ledge and configured to support the button, the support block configured to move within the support ledge to allow the moving mechanism to move between the open position and the closed position; and a locking ledge configured to maintain a position of a hook on the button when the hook contacts the locking ledge to lock the suture device in the closed position.

In another embodiment, the techniques described herein relate to a method of operating a suture device. The method includes providing a suture device. The suture device includes: a first jaw and a second jaw disposed adjacent to one another and connected by a pivot; an arm connected to the second jaw and the pivot; a moving mechanism disposed adjacent to the arm. The moving mechanism includes a button, a support block, and a locking ledge. The method further includes moving, by a user, the moving mechanism from an open position to a closed position by applying pressure to the button to compress the arm and close the first jaw and the second jaw; locking the suture device in the closed position by sliding the button back to contact a hook on the button with the locking ledge; and unlocking the suture device by further pressing the button and sliding the button forward into the open position,

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C are top perspective views of a first embodiment of a suture device, in accordance with the present technology;

FIG. 2 is a top, front perspective sectional view of the first embodiment of the suture device in an open position;

FIG. 3 is a top, rear perspective sectional view thereof;

FIG. 4 is a top perspective view thereof;

FIG. 5 is a top, front perspective sectional view of the first embodiment of the suture device in a closed position,

FIG. 6 is a top, rear perspective sectional view thereof;

FIG. 7A is a top perspective view of a second embodiment of the suture device in an open position, in accordance with the present technology;

FIG. 7B. is a top perspective view of the second embodiment of the suture device in a closed position;

FIG. 8 depicts a needle driver of the prior art along with the first and second embodiments of the suture device constructed in accordance with the present technology;

FIG. 9 is a side view of another embodiment of the suture device, in accordance with the present technology;

FIG. 10 is a cross sectional view of an embodiment of the suture device, in accordance with the present technology;

FIGS. 11A-11C is a cross sectional view of alternative embodiment of the suture device, in accordance with the present technology; and

FIG. 12 is a flowchart illustrating a method of operating a suture device, according to some embodiments of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom.

Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present, a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

All documents, including patents, patent applications, and scientific literature cited in this detailed description are incorporated herein by reference, unless otherwise expressly indicated. Where any conflict or ambiguity can exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that can be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it can be directly on, engaged, connected or coupled to the other element or layer, or intervening elements or layers can be present. In contrast, when an element is referred to as being “directly on,” “directly engaged to,” “directly connected to” or “directly coupled to” another element or layer, there can be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. can be used herein to describe various elements, components, regions, layers and/or sections, these elements, components, regions, layers and/or sections should not be limited by these terms. These terms can be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, can be used herein for ease of description to describe one element or feature's relationship to another element(s) or feature(s) as illustrated in the figures. Spatially relative terms can be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

The present technology improves upon standard disposable needle drivers and suture scissors used for wound closure. The present invention incorporates both cutting and needle driving features into a single ergonomic device to address the problems currently associated with hand and soreness attributable to prolonged use of such devices. Specifically, the enhanced grip, locking mechanism, and built-in scissors improve comfort, control, and efficiency compared to existing disposable suturing tools.

In some embodiments, the method can include cutting an object disposed between a first cutting surface on a first jaw and a second cutting surface on a second jaw when the first jaw and the second jaw can be closed. In some embodiments, moving a sliding mechanism from an open position to a closed position can include placing a spring disposed adjacent to an arm in tension. In some embodiments, locking the suture device can include moving a hook and the button axially relative to a longitudinal axis of the suture device.

In some embodiments, the method can include allowing the suture device to lock in both the open position and the closed position using a sliding mechanism disposed between the second jaw and a support ledge. In some embodiments, the suture device can further include a hand grip shaped to ergonomically fit in a hand of the user to allow the user to comfortably hold the suture device. In some embodiments, the user can hold the suture device for an extended period of time while operating the suture device. In some embodiments, moving the moving mechanism can include relaxing a spring when moving to the open position. In some embodiments, moving the moving mechanism can include compressing an arm connected to the second jaw and the pivot. In some embodiments, the suture device can be formed of plastic.

In some embodiments, the jaws of the suture device can be removed and replaced, enabling warn out surfaces of the suture device to be replaced without having to replace the entire suture device. The suture device can also include modular disposable tips to accommodate different suturing needs. Examples of such tips include, but are not limited to, blunt plastic tips, tapered metal tips, and wide grasping platforms. Such modular tips can either replace the jaws of the suture device or can clip onto the cutting portion of the jaws. The modularity of the tips can enable a single suture device to be utilized for multiple purposes and thus can expand the usefulness of the suture device for diverse lacerations and procedures. The present invention can also incorporate both cutting and needle driving features into a single ergonomic device, whereas the standard equipment uses separate tools for cutting (e.g. straight scissors) and needle driving.

In some embodiments, the suture device can also include suture feed technology. The suture feed technology can be integrated within the suture device to pass suture material through the jaws with the press of a lever or button. The suture feed technology can further enhance one-handed use and efficiency of the suture device.

In another embodiment, the suture device can include wireless connectivity.

Examples of wireless connectivity include wi-fi, 5G, and Bluetooth® wireless technology, and any other wireless technologies having similar capabilities. The wireless connection can allow device settings and usage data to be monitored remotely, for example through a smartphone app. The wireless technology allows the user to be provided with real-time information regarding device settings. Examples of such device setting include, but are not limited to, grip pressure, blade sharpness, and sterilization status, which can improve precision, while data helps prevent overuse injuries.

In some embodiments, the cutting surfaces and/or scissors can utilize ultrasonic or other similar vibrations, as are known to those of skill in the art can be used to improve cutting ability, thereby reducing strain on the blades over extended procedures. The inclusion of vibrations technology can help to eliminate friction, thus allowing smooth, precise cutting.

In some embodiments, the suture device can also include an OLED status display. The OLED can provide the user of the suture device with device related information. Some non-limiting examples of related information include, but are not limited to, grip pressure, lifetime use statistics, sterilization confirmation, and procedure time to assist documentation and prevent errors.

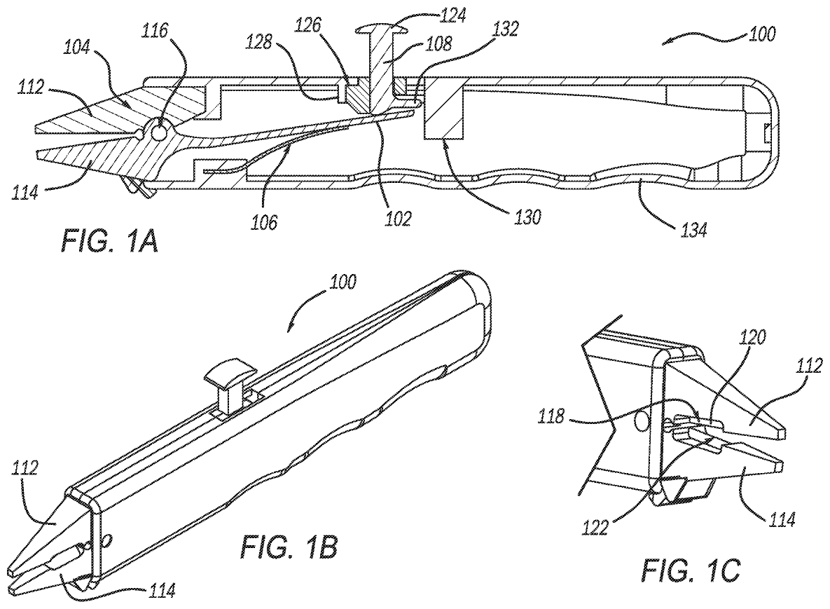

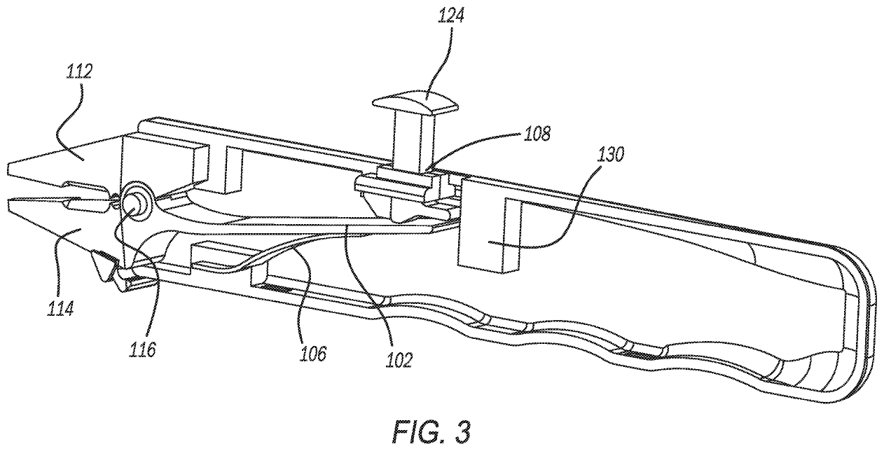

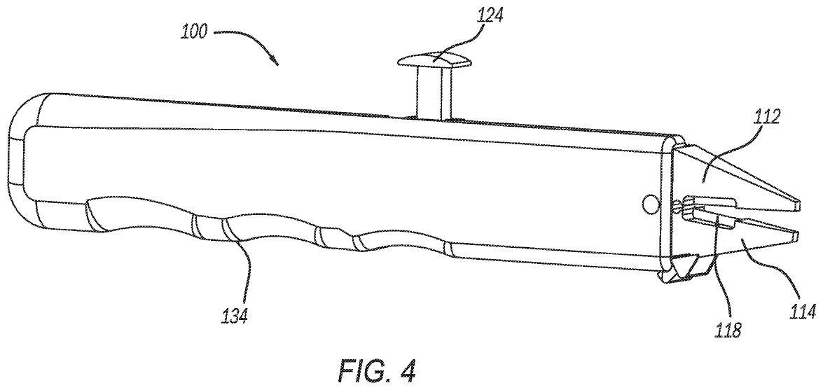

Referring now to the Figures, the present technology relates to a suture device shown generally at 100 in accompanying FIGS. 1-11. Advantageously, the suture device 100 can allow for reliable and consistent suturing by a user. The ergonomic design of suture device 100 can allow for the user to hold the suture device 100 with either their left hand or their right hand for an extended period of time without causing hand cramping. Additionally, the gripping and locking features can allow for the certified medical professional to have more range of motion while using the suture device 100. As shown in FIGS. 1A-1C, the suture device 100 can include an arm 102, a pair of jaws 104, a spring 106, and a moving mechanism 108 contained within the suture device 100.

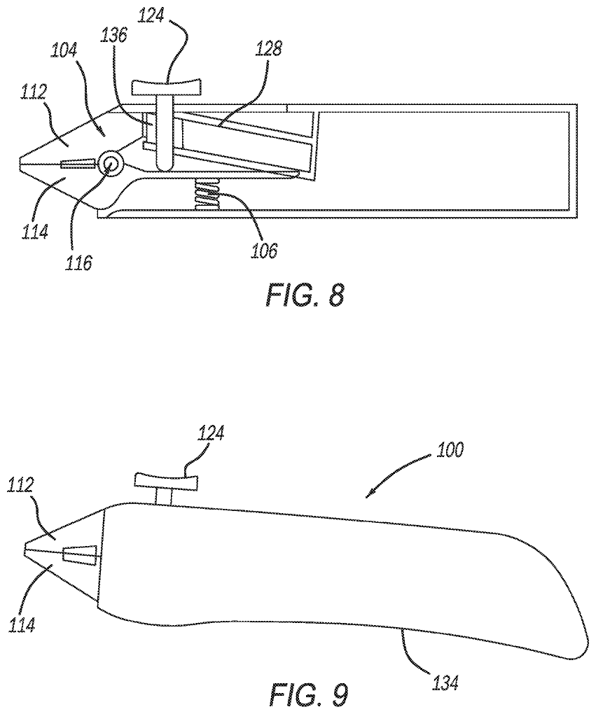

The button 124 can also include a lubricant to prevent binding of the button 124 during use. Examples of acceptable lubricants are known to those of skill in the art. In an embodiment shown in FIGS. 1A and 1C, the suture device 100 can include the pair of jaws 104. The pair of jaws 104 can further include a first jaw 112 and a second jaw 114. The first jaw 112 and the second jaw 114 can be disposed adjacent to one another and can be connected by a pivot 116. The second jaw 114 and the pivot 116 can be disposed on an arm 102. The moving mechanism 108 can be disposed adjacent to the arm 102 and can be configured to move between an open position 151, shown in FIG. 7A, and a closed position 152, shown in FIG. 7B. The pair of jaws 104 can be formed of any rigid material. Further, the length of the jaws 104 can vary depending on the intended use of the jaws, and can be optimized by one of skill in the art. As a non-limiting example, the pair of jaws 104 can be formed of machine spring steel. One of ordinary skill in the art can select a suitable material for the pair of jaws 104. The button 124 can also include a lubricant to prevent binding of the button 124 during use. Examples of acceptable lubricants are known to those of skill in the art.

With reference to FIG. 1C, the pair of jaws 104 can include a cutting portion 118 formed by a first cutting surface 120 on the first jaw 112 and a second cutting surface 122 on the second jaw 114. The first cutting surface 120 and the second cutting surface 122 can be configured to cooperate to cut in operation such that the first jaw 112 and the second jaw 114 can contact and the cutting action can occur. Advantageously, this can allow the certified medical professional to grip an object as thin as, for example, a suture, and cut the suture. As a non-limiting example, the first cutting surface 120 and the second cutting surface 122 can be any combination of at least one of blade, a shear, and a flat. For example, the cutting portion 118 can be one blade and one shear, two blades in shear, a blade and flat with tight tolerances, or two blades that meet with tight tolerances. Desirably, the cutting portion 118 can allow for the user to easily cut a material (e.g., a suturing material) without having to use another device, such as scissors. The cutting portion 118 can be used in the same manner as operating the pair of jaws 104 and the pivot 116.

As shown in FIGS. 1-3, the moving mechanism 108 can be disposed adjacent to the arm 102. With continued reference to FIGS. 1-3, the moving mechanism 108 can include a button 124, a support block 126, a support ledge 128, and a locking ledge 130. The button 124 can be disposed adjacent to and in contact with the arm 102. The button 124 can be configured to compress the arm 102. The button 124 can be configured to move through the support block 126 and the support ledge 128. The button 124 can include a hook 132 configured to contact the locking ledge 130. Where the hook 132 contacts the locking ledge 130, the locking ledge 130 can maintain the position of the book 132 until the user moves the hook 132 from the locking ledge 130. Advantageously, this can allow the user to lock the suture device 100 in the closed position. For example, in addition to radial movement of the button 124 relative to a longitudinal axis of the suture device 100, the button 124 can move axially, allowing the hook 132 to move axially relative to the locking ledge 130 to lock and unlock the suture device 100 in the closed position.

As shown in FIG. 1, the support block 126 can be disposed adjacent to the support ledge 128 and configured to support the button 124. The support block 126 can be configured to move within the support ledge 128 further allowing the moving mechanism 108 to move between the open position and the closed position. Where the moving mechanism 108 is in the open position, shown in FIG. 1, the spring 106 can be relaxed therefore allowing the arm 102 to be raised and the second jaw 114 to drop. As such, the pair of jaws 104 can be open. Advantageously, in the open position the user can prepare to grasp any object necessary.

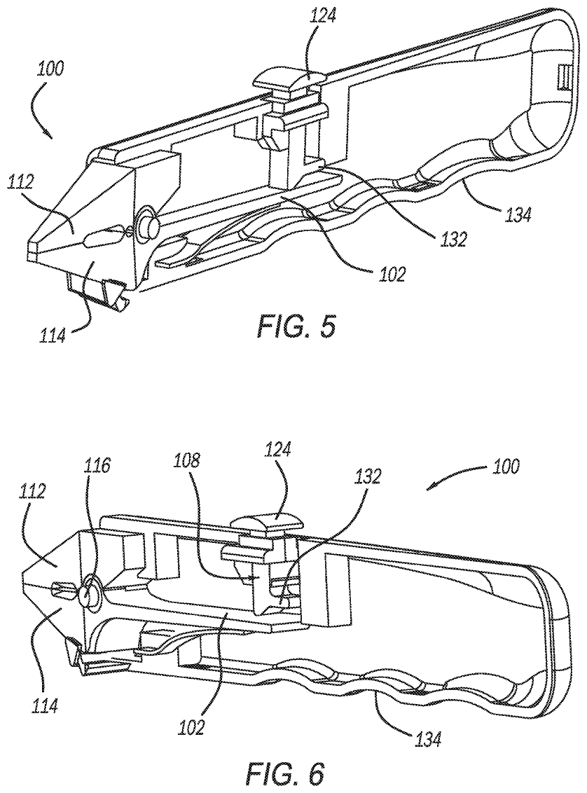

As shown in FIGS. 5-6 and 7B, the moving mechanism 108 can be in the closed position. In order to move the button 124 from the open position to the closed position, the user can apply pressure to the button 124 therefore moving the arm 102 down and compressing the spring 106. Where the moving mechanism 108 is in the closed position, the button 124 can be depressed by the user. Where the button 124 is depressed, the spring 106 can be depressed thereby placing it in tension and moving the second jaw 114 to close. Accordingly, the pair of jaws 104 can be in the closed position. Advantageously, in the closed position the pair of jaws 104 can be configured to hold an object between the first jaw 112 and the second jaw 114.

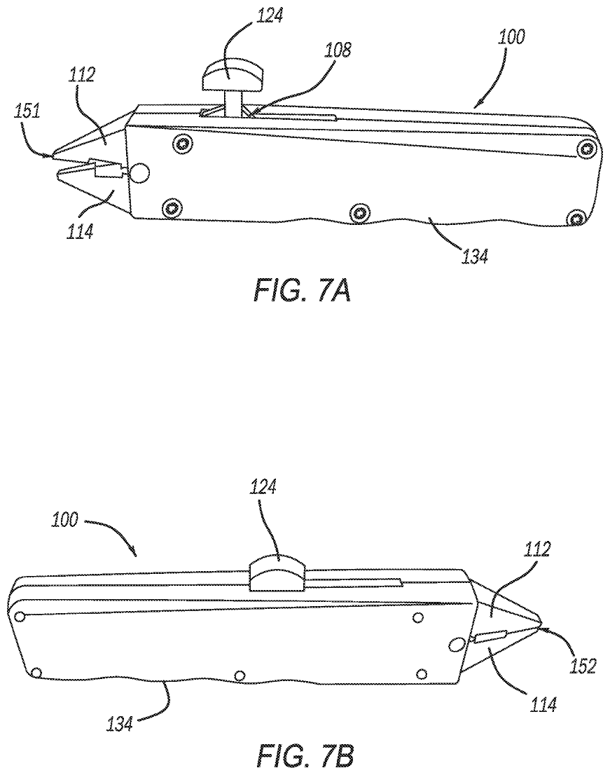

With continued reference to FIGS. 5-6, where the moving mechanism 108 is in the closed position, the moving mechanism 108 can be locked. The user can apply pressure to compress the button 124 and retain the closed position by sliding the button 124 back therefore contacting the hook 132 to the locking ledge 130. When the user wishes to move the moving mechanism 108 from the closed position while locked, the user can further depress the button 124 and slide the button 124 forward into the open position.

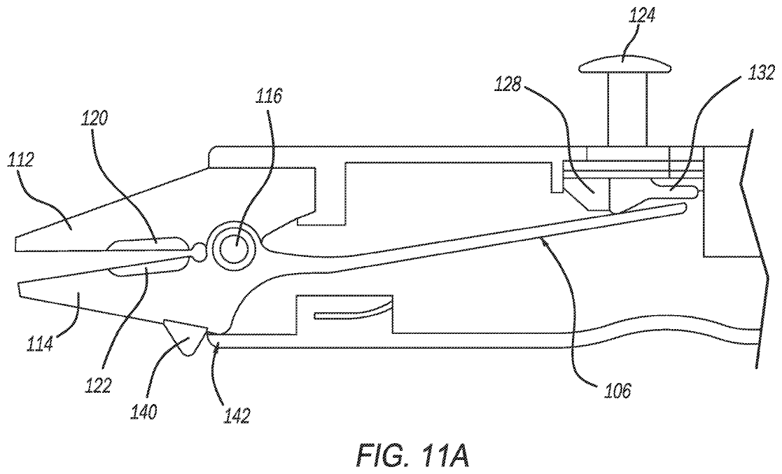

As shown in FIGS. 1A-1C, the arm 102 can be shaped to fit within the suture device 100. The arm 102 can be connected to the second jaw 114 and the pivot 116 and further disposed adjacent to the spring 106 and the button 124. Advantageously, and as described above, the arm 102 can provide a mechanism for allowing the button 124 to move the pair of jaws 104 between the open position and the closed position. The suture device 100 can also include a hand grip 134. The hand grip 134 can be ergonomically shaped to fit in the hand of the user and remain in the hand of the user for continued use over a long period of time, such as surgery. The hand grip 134 can be customizable with removable sleeves to fit small, medium, and large hands. Various sleeve materials like gel, rubber, or foam can also be used to provide personalized comfort and control. Advantageously, this can provide the user with the ability to comfortably hold the suture device 100 for extended periods of time. In an alternative, as shown in FIG. 9, the grip 134 of suture device 100 can provide a curved surface area for the user to grasp while in use.

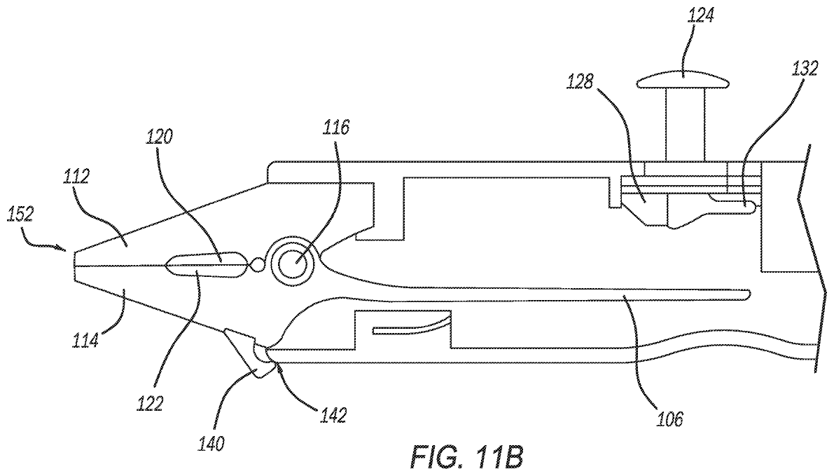

In another embodiment shown in FIG. 10, the suture device 100 can include a sliding mechanism 136 disposed between the second jaw 114 and the support ledge 128. The sliding mechanism 136 can allow the suture device 100 to be locked in the open position and the closed position. Where the sliding mechanism 136 is in the open position, the sliding mechanism 136 can move toward a rear end 138 of the suture device 100, therefore moving the second jaw 114 away from the first jaw 112. The trajectory of the second jaw 114 moving away from the first jaw 112 can be seen in FIG. 9.

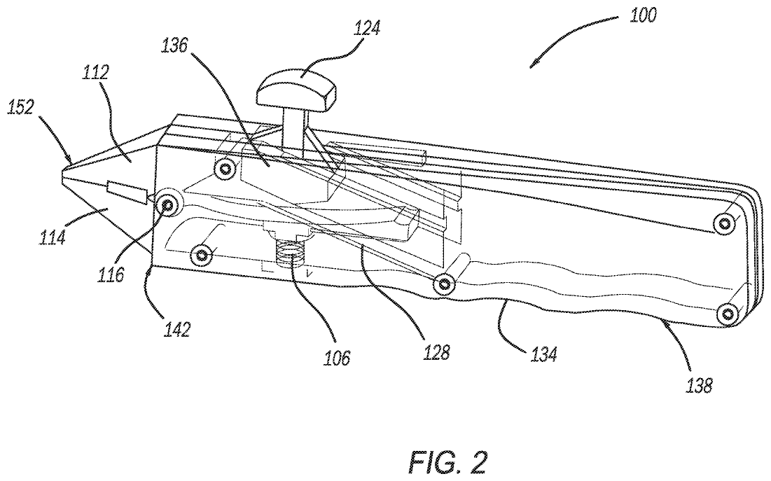

In another embodiment, as shown in FIG. 2, the support ledge 128 can be formed as a track like system, within which the support block 126 can sit. This can allow for increased clamp load within the suture device 100.

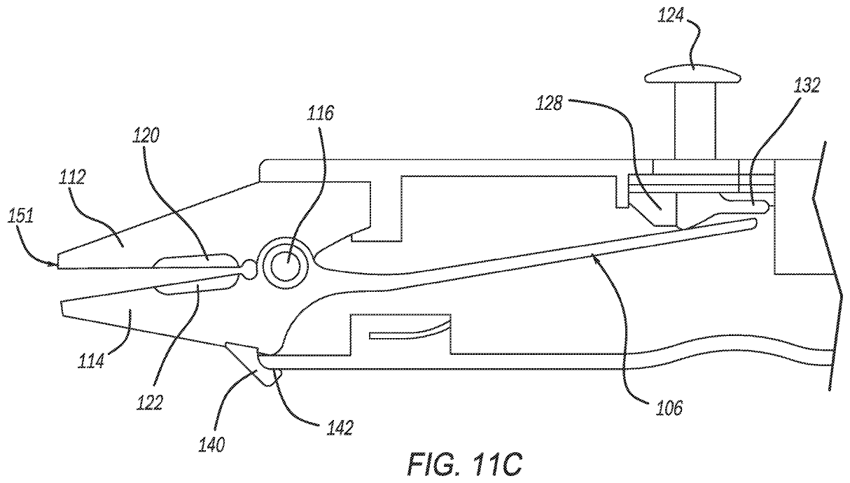

In another embodiment, as shown in FIGS. 11A-11C, the suture device 100 can include a notch 140. The notch 140 is disposed on opposite the second cutting surface 122 on the second jaw 114 and in close proximity to insertion end 142 of the pair of jaws 104 into the suture device 100. The notch can be designed to aid in holding and/or positioning the suture during use. The notch 140 can be a formed of the same material as the pair of jaws 104, as a feature of the jaws 104, or another biocompatible materials, examples of which are generally known to those of skill in the art. The notch 140 can be formed into a shape known to those of skill in the art to function in the desired manner. Some non-limited examples include, but are not limited to, a triangular piece of material (FIG. 11A), a hook that can partially engage the insertion end 142 while creating a gap within which the notch 140 can hold the thread (FIG. 11C), and a hook that can completely engage the insertion end 142 (FIG. 11C).

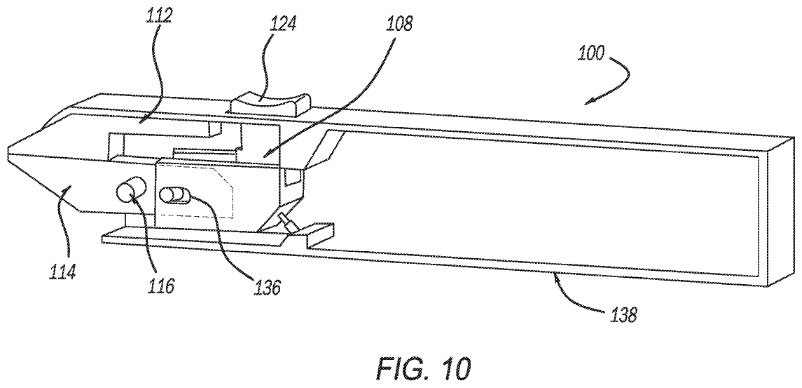

The suture device 100 can be formed of any rigid material. As a non-limiting example, the suture device 100 can be formed of a plastic material such as polycarbonate or polypropylene. Alternatively, the suture device 100 can be formed of a disposable plastic material. One of ordinary skill in the art can select other suitable materials for the suture device 100 to be formed of within the scope of the present disclosure. The suture device can have a width (W) and a length (L), shown in FIG. 8, to allow for the suture device 100 to be easily held in the hand of the user. As a non-limiting example, the width (W) can be about ½ inch, and the length (L) can be about 5 inches. One of ordinary skill can select a suitable length (L) and width (W) of the suture device 100 within the scope of the present disclosure. Additionally, the button 124 can be positioned along the length (L) of the suture device 100. As a non-limiting example, the button can move between a length (L) of about 1 inch to about 2 inches along the suture device 100 as the button 124 moves between the open position and the closed position. Alternatively, the size of the suture device 100 depending on the hand size of the intended user. For example, the suture device 100 can be provided in a small, medium, or large size, wherein each of the sizes is formed of dimensions, as are known to those of skill in the art, and therefore can provide roughly three differently sized suture devices.

In use, the suture device 100 functions with the following general steps. The button 124 can be depressed with the thumb of the user to cut suture or grip needle. The button 124 can subsequently be pulled back and locked into place. The suture device 100 can be used at any angle or orientation and can cut a range of thread.

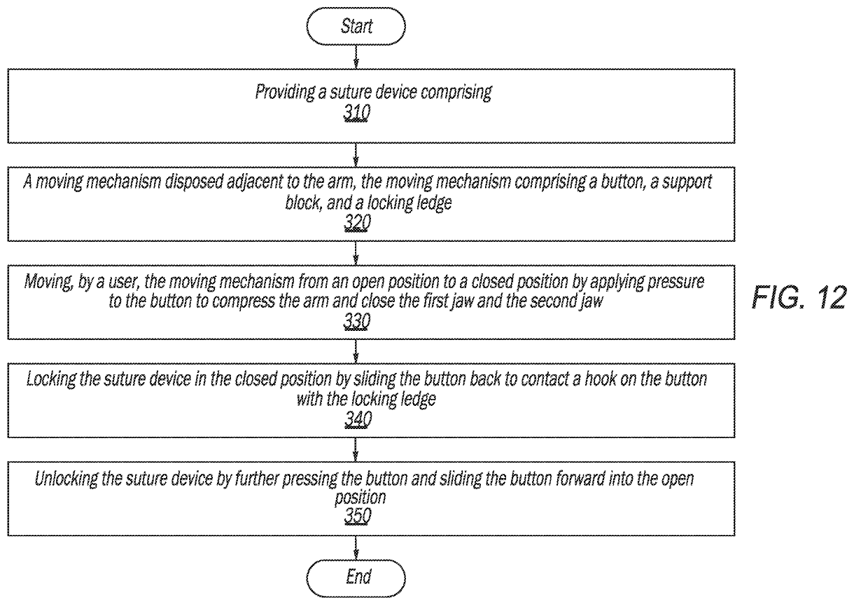

FIG. 12 is a flowchart that describes a method of operating a suture device, according to some embodiments of the present disclosure. In some embodiments, at step 310, the method can include providing a suture device comprising. At step 330, the method can include moving, by a user, the moving mechanism from an open position to a closed position by applying pressure to the button to compress the arm and close the first jaw and the second jaw.

At 340, the method can include locking the suture device in the closed position by sliding the button back to contact a hook on the button with the locking ledge. As shown at step 350, the method can include unlocking the suture device by further pressing the button and sliding the button forward into the open position. At 320, the providing can include a moving mechanism disposed adjacent to the arm, the moving mechanism comprising a button, a support block, and a locking ledge. A first jaw. A pivot. A second jaw disposed adjacent to the first jaw and connected by the pivot. An arm connected to the second jaw and the pivot.

### EXAMPLES

Example embodiments of the present technology are provided with reference to the several figures enclosed herewith.

Example 1: In use, the suture device can be used to suture lacerations in an emergency room setting. The ergonomic grip and built-in scissors reduce hand strain for physicians suturing multiple patient lacerations during a shift.

Example 2: The suture device can also be used in field surgery and mobile medical units to quickly suture wounds with limited equipment available. The locking mechanism provides reliable wound closure even in turbulent environments.

Example 3: Plastic surgeons can utilize the suture device for cosmetic surgery incisions such as facelifts and tummy tucks. The ergonomic design can allow precise suturing over long procedures without hand fatigue.

Example 4: Veterinarians can use the disposable suture device for closing surgical incisions in animals. The one-handed operation leaves the other hand free to control or comfort anxious pets.

Example 5: Nurses can conveniently practice suturing techniques using the device for training purposes. The similar functionality to traditional tools helps build skills with enhanced comfort.

Example 6: First responders can carry the compact suture device in mobile kits to rapidly stabilize lacerations at emergency sites until further medical aid arrives. The simple design is intuitive even for those with minimal suturing experience.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A suture device comprising:
a first jaw and a second jaw disposed adjacent to one another and connected by a pivot;
an arm connected to the second jaw and the pivot; and
a moving mechanism disposed adjacent to the arm and configured to move between an open position and a closed position, the moving mechanism comprising:
a button disposed adjacent to and in contact with the arm, the button configured to compress the arm;
a support block disposed adjacent to a support ledge and configured to support the button, the support block configured to move within the support ledge to allow the moving mechanism to move between the open position and the closed position; and
a locking ledge configured to maintain a position of a hook on the button when the hook contacts the locking ledge to lock the suture device in the closed position.

2. The suture device of claim 1, wherein the first jaw and the second jaw comprise a cutting portion formed by a first cutting surface on the first jaw and a second cutting surface on the second jaw, the first cutting surface and the second cutting surface configured to cooperate to cut in operation.

3. The suture device of claim 2, wherein the cutting portion comprises at least one of a blade, a shear, and a flat.

4. The suture device of claim 1, wherein the button is configured to move radially relative to a longitudinal axis of the suture device and configured to move axially to allow the hook to move axially relative to the locking ledge to lock and unlock the suture device in the closed position.

5. The suture device of claim 1, further comprising a spring disposed adjacent to the arm, wherein when the moving mechanism is in the open position, the spring is relaxed allowing the arm to raise and the second jaw to drop so that the first jaw and the second jaw are open.

6. The suture device of claim 5, wherein when the moving mechanism is in the closed position, the button is depressed by a user thereby placing the spring in tension and moving the second jaw to close so that the first jaw and the second jaw are in the closed position.

7. The suture device of claim 1, further comprising a hand grip ergonomically shaped to fit in a hand of a user.

8. The suture device of claim 1, further comprising a sliding mechanism disposed between the second jaw and the support ledge configured to allow the suture device to be locked in the open position and the closed position.

9. The suture device of claim 1, formed of plastic.

10. A method of operating a suture device, the method comprising:
providing a suture device comprising: a first jaw and a second jaw disposed adjacent to one another and connected by a pivot; an arm connected to the second jaw and the pivot;
a moving mechanism disposed adjacent to the arm, the moving mechanism comprising a button, a support block, and a locking ledge;
moving, by a user, the moving mechanism from an open position to a closed position by applying pressure to the button to compress the arm and close the first jaw and the second jaw;
locking the suture device in the closed position by sliding the button back to contact a hook on the button with the locking ledge; and
unlocking the suture device by further pressing the button and sliding the button forward into the open position.

11. The method of claim 10, further comprising cutting an object disposed between a first cutting surface on the first jaw and a second cutting surface on the second jaw when the first jaw and the second jaw are closed.

12. The method of claim 10, wherein moving the moving mechanism from the open position to the closed position comprises placing a spring disposed adjacent to the arm in tension.

13. The method of claim 10, wherein locking the suture device comprises moving the hook and the button axially relative to a longitudinal axis of the suture device.

14. The method of claim 10, further comprising allowing the suture device to lock in both the open position and the closed position using a sliding mechanism disposed between the second jaw and the support block.

15. The method of claim 10, wherein the suture device further comprises a hand grip shaped to ergonomically fit in a hand of the user to allow the user to comfortably hold the suture device.

16. The method of claim 15, wherein the user holds the suture device for an extended period of time while operating the suture device.

17. The method of claim 10, wherein moving the moving mechanism comprises relaxing a spring when moving to the open position.

18. The method of claim 10, wherein moving the moving mechanism comprises compressing an arm connected to the second jaw and the pivot.

19. The method of claim 10, wherein the suture device is formed of plastic.

20. A suture device comprising:
a first jaw and a second jaw disposed adjacent to one another, the first jaw being fixed and the second jaw connected by a pivot;
an arm connected to the second jaw and the pivot; and
a moving mechanism disposed adjacent to the arm and configured to move between an open position and a closed position, the moving mechanism comprising:
a button disposed adjacent to and in contact with the arm, the button configured to compress the arm;
a support block disposed adjacent to a support ledge and configured to support the button, the support block configured to move within the support ledge to allow the moving mechanism to move between the open position and the closed position; and
a locking ledge configured to mate with the button when the button is compressed to lock the suture device in the closed position.
